(19) United States
(12) Patent Application Publication
(10) Pub. No.: US 2021/0121299 A1
(43) Pub. Date: Apr. 29, 2021

(54) SPINAL IMPLANT DEVICE

(71) Applicant: CTL Medical Corporation, Addison, TX (US)

(72) Inventor: Zeshan Hyder, Munster, IN (US)

(21) Appl. No.: 16/664,279

(22) Filed: Oct. 25, 2019

(51) Int. Cl. A61F 2/44

(52) U.S. Cl. CPC A61F 2/447; A61F 2002/30556; A61F 2002/4475

(57) ABSTRACT

Disclosed are devices for the fixation and support of vertebrae, particularly spinal implant devices having adjustability.

### FIELD OF THE INVENTION

The present subject matter relates generally to devices for the fixation and support of vertebrae. In particular, the present subject matter relates to an implant device having adjustability.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

The spinal curvature disorders and/or contour issues present on the surfaces of the vertebrae may present additional challenges. As such, there is need for further improvement, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with an aspect of the present subject matter, an implant device for the spine is provided. The implant device is for location between two adjacent vertebrae. The implant device includes: a first engagement member configured to interface with a first of the two adjacent vertebrae, the first engagement member including plural engagement areas; a second engagement member configured to interface with a second of the two adjacent vertebrae, the second engagement member including plural engagement areas, distance exists between respective engagement areas of the first and second engagement members; and means for independently adjusting the distance between at least some of the respective engagement areas.

In accordance with another aspect of the present subject matter, an implant device for the spine is provided. The implant device is for location between two adjacent vertebrae. The implant device includes: a first engagement member configured to interface with a first of the two adjacent vertebrae, the first engagement member including plural corner areas; a second engagement member configured to interface with a second of the two adjacent vertebrae, the second engagement member including plural corner areas, distance exists between respective corner areas of the first and second engagement members; and means for independently adjusting the distance between at least some of the respective corner areas.

In accordance with another aspect of the present subject matter, an implant device for the spine is provided. The implant device is for location between two adjacent vertebrae. The implant device includes: a first engagement member configured to interface with a first of the two adjacent vertebrae, the first engagement member including plural side areas; a second engagement member configured to interface with a second of the two adjacent vertebrae, the second engagement member including plural side areas, distance exists between respective side areas of the first and second engagement members; and means for independently adjusting the distance between at least some of the respective side areas.

In accordance with another aspect of the present subject matter, an implant device for the spine is provided. The implant device is for location between two adjacent vertebrae. The implant device being adaptive to address at least one spine curvature disorder. Within one example, the at least one spine curvature disorder includes Lordosis.

In accordance with another aspect of the present subject matter, an implant device for the spine is provided. The implant device is for location between two adjacent vertebrae. The two adjacent vertebrae have opposed faces that have at least one opposing inconsistency, the implant device being adaptive to address the at least one opposing inconsistency.

In accordance with another aspect of the present subject matter, an implant device for the spine is provided. The implant device is for location between two adjacent vertebrae. The implant device includes: a first engagement member configured to interface with a first of the two adjacent vertebrae, the first engagement member including plural engagement areas; a second engagement member configured to interface with a second of the two adjacent vertebrae, the second engagement member including plural engagement areas, distance exists between respective engagement areas of the first and second engagement members; and at least one mechanism that is operable to independently adjusting the distance between at least some of the respective engagement areas. Within one example, the at least one mechanism includes at least one screw.

In accordance with another aspect of the present subject matter, a method for manufacturing an implant device as indicated above is provided.

In accordance with another aspect of the present subject matter, a method for manufacturing an implant device as set for within any of the details described with the present application is provided.

In accordance with another aspect of the present subject matter, an implant device for the spine as set for within any of the details described with the present application is provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 is a perspective view of an example implant device in accordance with at least one aspect of the present subject matter, and shows one relative position of two engagement members;

FIG. 2 is a front view of the implant device of FIG. 1, and shows another relative position of the two engagement members;

FIG. 3 is an enlarged front view of the implant device of FIG. 1, and shows another relative position of the two engagement members;

FIG. 4 is another perspective view of the implant device of FIG. 1, and shows another relative position of the two engagement members;

FIG. 5 is another perspective view of the implant device of FIG. 1, and shows another relative position of the two engagement members;

FIG. 6 is a lateral view of the implant device of FIG. 1, and shows another relative position of the two engagement members;

FIG. 7 is a lateral view of the implant device of FIG. 1, and shows another relative position of the two engagement members;

FIG. 8 is a partially torn-open perspective view of the implant device of FIG. 1, showing various details of an adjustment mechanism to independently adjusting a distance between at least some of respective engagement areas of the two engagement members;

FIG. 9 is a further partially torn-open perspective view of the implant device of FIG. 1, showing various details of another exemplary mechanism for independently adjusting a distance between at least some of respective engagement areas of the two engagement members;

FIG. 10 is another perspective view of the implant device of FIG. 1, and shows another relative position of the two engagement members;

FIG. 11 is a front view of an implant device in accordance with at least one aspect of the present subject matter;

FIG. 12 is a partially torn-open perspective view of the implant device of FIG. 11;

FIG. 13 is a lateral view of the implant device of FIG. 11;

FIG. 14 is a perspective view of the implant device of FIG. 11;

FIG. 15 is partially torn-open perspective view another exemplary embodiment of an implant device in accordance with at least one aspect of the present subject matter, showing an alternative adjustment plate to independently adjusting a distance between at least some of respective engagement areas of the two engagement members;

FIG. 16 is a schematized perspective view of wedge and ball members for independently adjusting a distance between at least some of respective engagement areas of the two engagement members;

FIG. 17 is a perspective view of one example engagement member that can be used within an implant device;

FIG. 18 is a perspective view of an example skirt member that can be used within an implant device; and

FIG. 19 is a lateral side view of an example spinal column within which an implant device of the subject matter can be used.

### DETAILED DESCRIPTION OF THE INVENTION

The present subject matter relates generally to devices for the fixation and support of vertebrae. In particular, the present subject matter relates to an implant device having adjustability. The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately.

Alternatively or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

The spinal curvature disorders and/or contour issues present on the surfaces of the vertebrae may present additional challenges. As such, there is need for further improvement. The present subject matter is such improvement. The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components are arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

The implant device and any portions or combination of portions thereof, such as those described and illustrated herein, can be constructed from radiopaque or radiolucent materials, other materials or combinations of such materials. Radiolucent materials can include, but are not limited to, polymers, carbon composites, fiber-reinforced polymers, plastics, combinations thereof and the like. One example of a radiolucent material that can be used with the present subject matter is PEEK-OPTIMA® polymer (commercially available from Invibio Inc., Greenville, S.C., USA). The PEEK-OPTIMA® polymer is a polyaromatic semicrystalline thermoplastic known generically as polyetheretherketone. The PEEK-OPTIMA® polymer is a biocompatible and inert material. Radiopaque materials are traditionally used to construct devices for use in the medical device industry. Radiopaque materials can include, but are not limited to, metal, aluminum, stainless steel, titanium, titanium alloys, cobalt chrome alloys, combinations thereof and the like.

Radiolucent materials can be utilized to facilitate radiographic evaluation of fusion material or vertebrae near an implant device. For example, radiolucent materials permit x-rays to pass through the implant device or components thereof so that developed x-ray pictures provide more visibility of the fusion material and vertebrae without significant interference, such as imaging artifacts, caused by the implant device. Radiolucent materials can enable clear visualization through imaging techniques such as x-ray and computer tomography (CT), whereas traditional radiopaque metallic or alloy materials can generate imaging artifacts or scatter that may prevent a comprehensive inspection of the surrounding tissue, vertebra and fusion material. In order to address the general disadvantage that some radiolucent materials lack the strength of radiopaque materials, design modifications may be required to provide adequate structural integrity and durability to the implant device. For example, the thickness of portions of the implant device subject to stress and strain can be increased in order to add support and structural integrity. Thicker or bulkier construction can mitigate the stresses of vertebra migration and toggling of the bone fasteners that may cause the implant device to bend, crack or otherwise be damaged while in use.

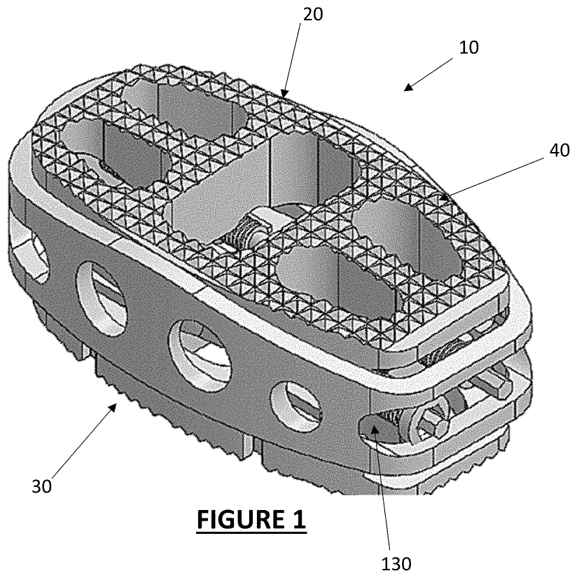

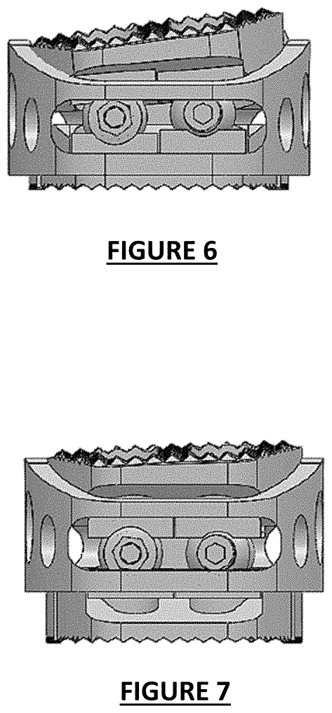

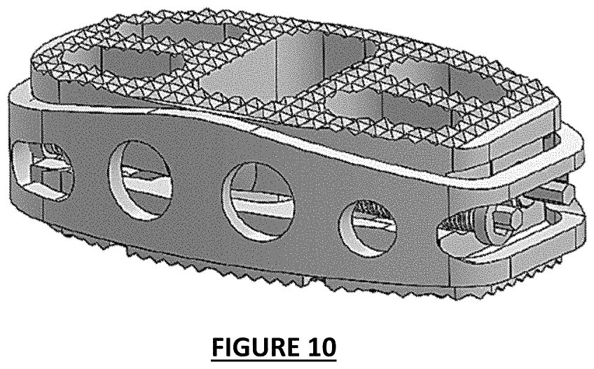

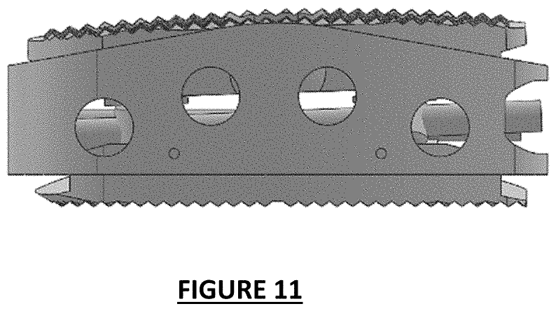

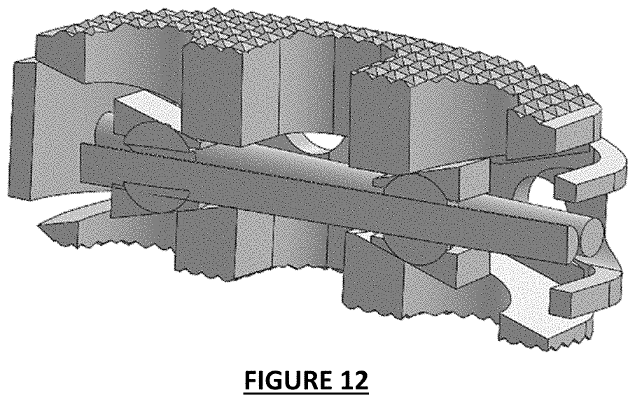

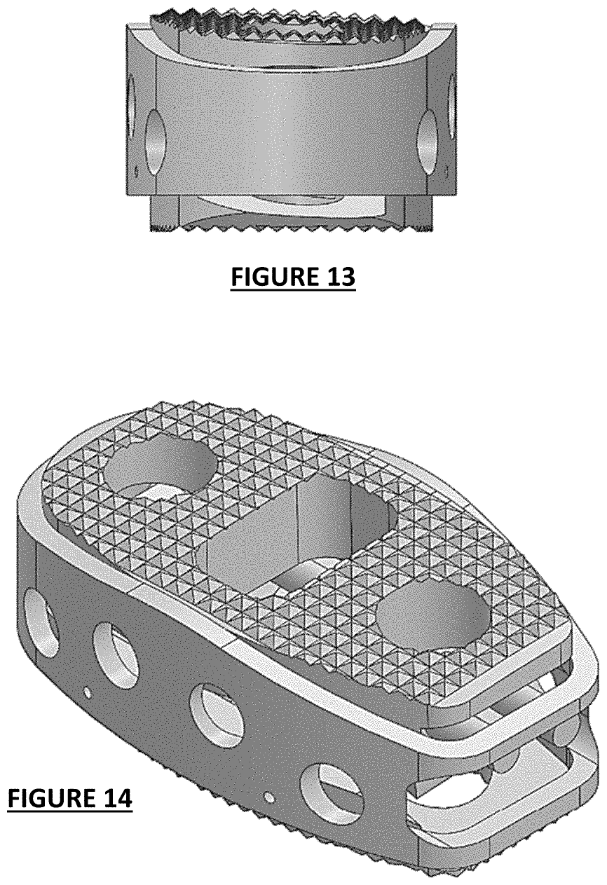

Referring initially to FIGS. 1 through 10, an implant device 10 that is presented is to be understood to be just one example embodiment. Other different embodiments are contemplated and are within the scope of this application. FIGS. 11-18 help emphasize such other different embodiments, and the broad scope of this application.

It is to be appreciated that the examples shown herein are suitable for lateral or postero-lateral insertion. However, it is to be appreciated that other configurations for other insertion directions are contemplated.

The implant device 10 illustrated in FIGS. 1 through 10 includes a first engagement member 20 and a second engagement member 30 (e.g., first and second) that are each configured to interface with a respective one (e.g., first and second) of two adjacent vertebrae. It is to be appreciated that each engagement member may have a textured engagement surface 40 that bears against the respective vertebra. The engagement surface may be textured in any suitable manner. The shown example has teeth-like projections. However, it is contemplated that other texturing is possible. For example, the texturing may mimic the texturing of natural bone surface. Such could be accomplished via 3-D material building (e.g., 3-D printing). Metals, such as titanium and stainless steel, or other any other material could be employed for such 3-D material building.

Each (e.g., first or second) engagement member may include plural engagement areas—such as wherein the engagement area can be divided as desired into a plurality of areas. The areas can be via any divisions. For example, the engagement areas could be four corner areas. As another example, the engagement areas could be four areas defined to be fore, aft, left lateral and right lateral. It is to be appreciated that the choice of division into engagement areas need not be an overall limitation upon the subject matter.

It is to be appreciated that there is respective distance between respective engagement areas of the first and second engagement members. For example, if the engagement areas are segregated into four corner areas, there is respective distance between respective first corner engagement areas of the first and second engagement members, respective distance between respective second corner engagement areas of the first and second engagement members, respective distance between respective third corner engagement areas of the first and second engagement members, and respective distance between respective fourth corner engagement areas of the first and second engagement members. At least some of these four distances can be independently adjusted. Another way of saying such is that at least some of these four distances can be adjusted to be different from at least some other of these four distances.

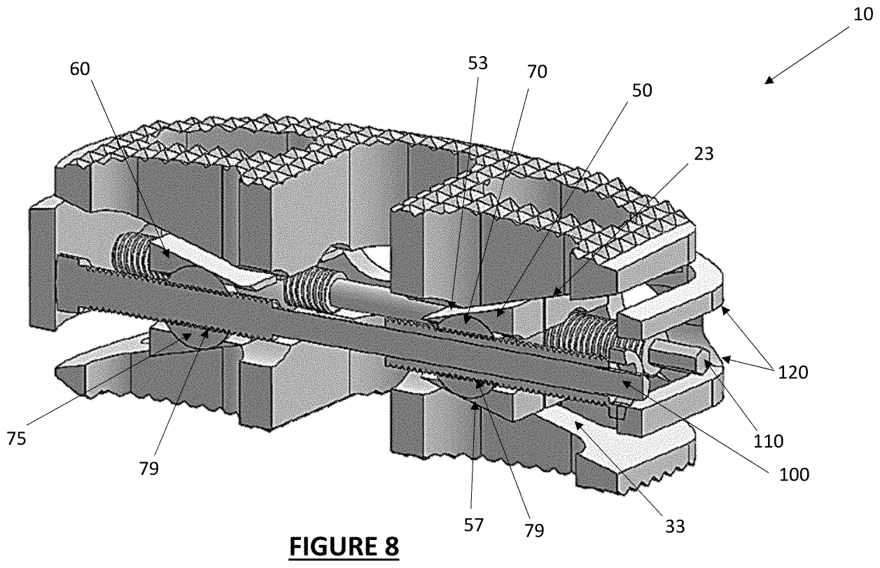

At least one adjustment mechanism/means is provided and is operable to independently adjusting the distance between at least some of the respective engagement areas. Within the example shown in FIG. 8, the adjustment mechanism can include first and second adjustment members 50 and 60, where each adjustment member 50 can include an upper wedging surface 53 and a lower wedging surface 57, which can interact with corresponding inclined surfaces 23 and 33 of the first and second engagement members 20 and 30. Desirably, as the adjustment members 50 and 60 are moved closer or further apart, the interaction of the wedging surfaces 53 and 57 with the inclined surfaces 23 and 33 will raise and/or lower the first and second adjustment members 50 and 60 relatively to each other, thereby increasing and/or decreasing the height of the implant 10. Also shown is a pair of spherical elements 70 and 75 (which can rotate within the adjustment members), each of which includes an internally threaded portion 79 which accommodates an externally threaded adjustment screw 100 (with a similar externally threaded adjustment screw 110 and associated components also shown) which operatively interacts with the first and second engagement members. This arrangement allows the adjustment members 50 and 60 to rotate and/or flex to some degree relative to each other, and also allows the first and second adjustment members 50 and 60 to assume a variety of non-planar configurations relative to each other and/or to the implant 10—allowing the implant to accommodate a wide range of anatomical variation.

In the disclosed embodiment, an enclosing skirt 120 is also shown which encircles, at least partially, the adjustment mechanism and the first and second engagement members. As disclosed, the skirt 120 can include one or more openings 130 (see FIG. 1) which provide access to the adjustment mechanism (i.e., the heads of the adjustment screws). However, it is contemplated that other/different structures/configurations for the skirt, the openings and/or the adjustment screw types and/or locations are contemplated. It is further contemplated that the encircling skirt could be omitted dependent upon the specific design of the implant device.

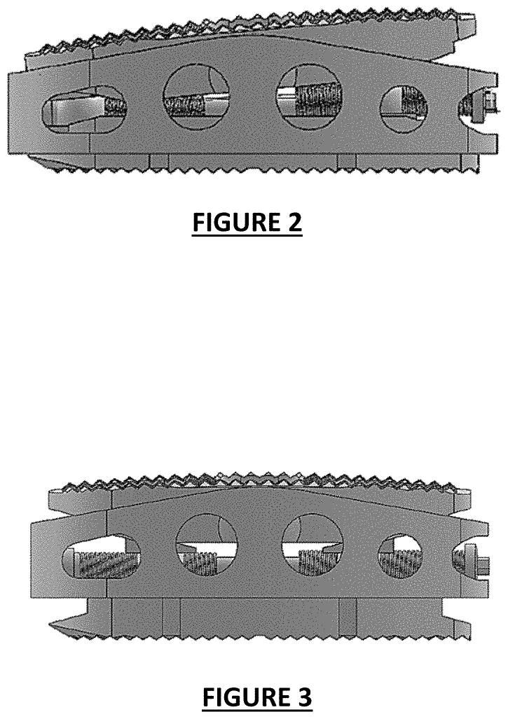

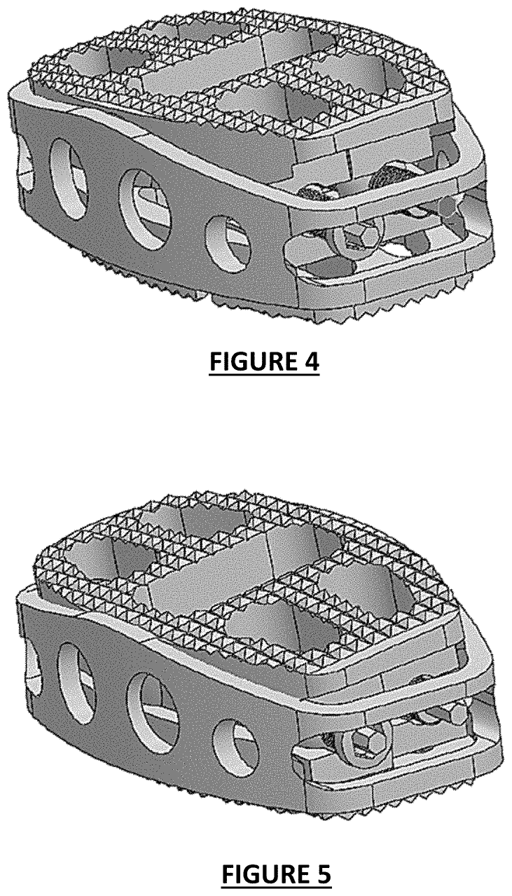

Note that with the various figures, different relative positions of the first and second engagement members are shown. In other words, different relative adjustment positions of the first and second engagement members can be accomplished via adjustment in separation and/or surface angulation of one of more of the first and second engagement members to achieve a variety of resulting implant shapes and/or sizes, thereby accommodating virtually any expected anatomical variation. For example, variation of the separation distance between the engagement members (i.e., without altering the angulation of the engagement members) can desirably cause an increase or decrease in the size or “height” of the implant, due to changes in the z-axis positioning of the implant components which engage the adjacent vertebrae. Concurrently, alterations in the “tilt angle” or angulation of one or both of the engagement surfaces of the engagement members in the medial-lateral (i.e., rotation about a y-axis) and/or anterior-posterior (i.e., rotation about an x-axis) axes of the implant will allow the implant to accommodate a wide variety of natural and/or surgically altered surfaces of the spine. For example, FIGS. 2 and 4 show some comparative medial-lateral (e.g., left-right) tilt, and FIGS. 5-7 show some comparative anterior-posterior (e.g., fore-aft) tilt. Moreover, various complex combinations (at various amounts) of comparative lateral (e.g., left-right) tilt and fore-aft (e.g., anterior-posterior) tilt can be accomplished, with or without concurrent adjustments in the height of the implant. In various embodiments, each respective engagement area (e.g., each corner) can have a different adjusted distance as compared to the other respective engagement areas (e.g., other corners). This can be referred to as independent adjustment of the various areas. Again, if different respective engagement areas are chosen/designated (e.g., fore, aft and two lateral areas), then those other respective engagement areas can be independently adjusted.

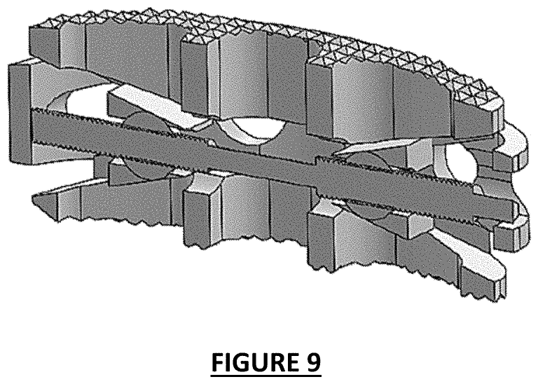

FIGS. 8 and 9, which are partial tear-way views, may provide the best viewing of an example of at least one mechanism that is operable to independently adjusting the distance between at least some of the respective engagement areas. Within the example, there are four wedge and ball/sphere arrangements near/adjacent to each of the four respective engagement areas (e.g., corners) of the first and second engagement members. Each wedge has a wedge surface that bears against a respective sloped/tapered surface located on an inner side of a respective one of the engagement members. Each ball/sphere (simply referred herein after as a ball) interacts with two respective wedges. The ball transfer motive force to the two respective wedges and also allows for any needed rotational orientation pivot adjustment. Each ball has a respective female threading within a bore extending into (or through) the ball.

A rotational threaded member of the adjustment mechanism has a matching/mating male threading that interacts with the female threading of one of the balls and thus can transfer motive force to the ball. As can be appreciated, via the cooperation of the threads (which in this embodiment can comprise a thread “pair” on each actuator screw having opposing thread directions, such that rotation of the screw in a clockwise direction causes the balls to approach each other and rotation in a counterclockwise direction causes the balls to move away from each other—or vica-versa), the ball and the wedges, rotational motive force on an actuator (i.e., by rotation of the hex “key” on the end of the adjustment screw 110), rotational motion can be translated into linear motive force to cause a relative linear movement between the wedges and the first and second engagement members. Moreover, because of the wedging action against the respective sloped/tapered surfaces located on the inner side of the first and second engagement members, the distance between the first and second engagement members, at the associated, respective engagement areas) is changed/adjusted.

Various configurations/constructions for rotational threaded member(s) that have the male threading(s) are contemplated. With the shown example of FIGS. 1-10, there are first-third rotational threaded members. Also, within the shown example, the first-third rotational threaded members extend for access regarding operational engagement to one lateral side of the device. It is contemplated that different access(es) regarding operational engagement is/are possible.

In some embodiments, such as shown in FIG. 8, the first rotational threaded member may have a long thin portion that extends through a hollow interior of the second rotational threaded member (i.e., a “composite” rotational threaded member). So, the first and second rotational threaded members may be co-axial in such embodiments, allowing the medial and lateral “balls” in some embodiments to be adjusted individually. In such embodiments, the first rotational threaded member may be adjusted using a male hex drive tool (not shown), while the second rotational threaded member may be adjusted using a female hex socket. A tool engagement head for each of the first and second rotational threaded members may be co-axial too and located at the lateral side for access. It is contemplated that a tool could be designed to engage/actuate only one of the first and second rotational threaded members at a time or engage/actuate both of the first and second rotational threaded members at the same time.

In at least one alternative embodiment, such as shown in FIG. 9), a third rotational threaded member may be utilized which includes a pair of balls which engage corresponding adjustment ramp elements. In such a design, the third rotational threaded member may be spaced apart and/or parallel to the second/first rotational threaded member, if desired.

In various other alternative embodiments, the adjustment mechanism for the spinal implant may incorporate a single threaded member in combination with a composite threaded member, such as shown FIGS. 8 and 9, or the adjustment member could incorporate a pair of single threaded members and/or a pair of composite threaded members, if desired

It is contemplated that the helix direction of the threaded portions of the first and second rotational threaded members could be similar or different. For example, the helix direction could be in the same direction or opposite directions. As such, rotating the first and second rotational threaded members in the same direction or in opposite directions could have different effects for adjusting the respective, associated distance between the respective, associated engagement areas of the first and second engagement members, as well as allow for individual adjustment of one ball.

The shown example presents the third rotational threaded member as having two threadings (e.g., two threaded portions or segments). The two threadings engaging two of the four internally-threaded balls. With such a configuration, the third rotational threaded member operates two of the four wedge and ball arrangements. Moreover, the operation of the two wedge and ball arrangements is simultaneous (i.e., rotation of the third rotational threaded member causes simultaneous actuation of the two wedge and ball arrangements so that distance change is simultaneously occurring at the two respective engagement areas of the first and second engagement members). Within one specific example, the distance changes at the two respective engagement areas of the first and second engagement members are in the same direction (e.g., both distances increase or decrease at the same time). Within one example, the simultaneously occurrence at the two respective engagement areas of the first and second engagement members is for addressing Lordosis.

Similar to the first and second rotational threaded members, a tool engagement head is located at the lateral side for access. Of course, different configurations/arrangements are contemplated.

With regard to the example, third rotational threaded member and associated two wedge and ball arrangements, the device is constructed/configured such that the two wedge and ball arrangements are permitted to have some ability to “float.” Specifically, the two wedge and ball arrangements can laterally shift relative to the first and second engagement members. This ability to “float” (e.g., shift) is useful to freely permit various canting angles between the one or more of the four respective engagement areas (e.g., corners).

It is to be appreciated that the skirt can be utilized to provide containment of the other components of the device. Also, it is to be appreciated that the skirt can use used to provide a one or more surface(s) (e.g., an inwardly facing surface) against which one of more of the rotational threaded member(s) may bear (e.g., at an end portion thereof). Within the shown example, the first rotational threaded member can engage and bear against the inwardly facing surface of the skirt. However, within the shown example, the third rotational threaded member need not bear against the skirt. Such non-bearing may be useful to provide the above-mentioned “floating.”

Of course, locking/securing mechanisms/means, if desired, are contemplated to help retain the device in a specific adjustment (e.g., at least some distance of the respective engagement areas are adjusted).

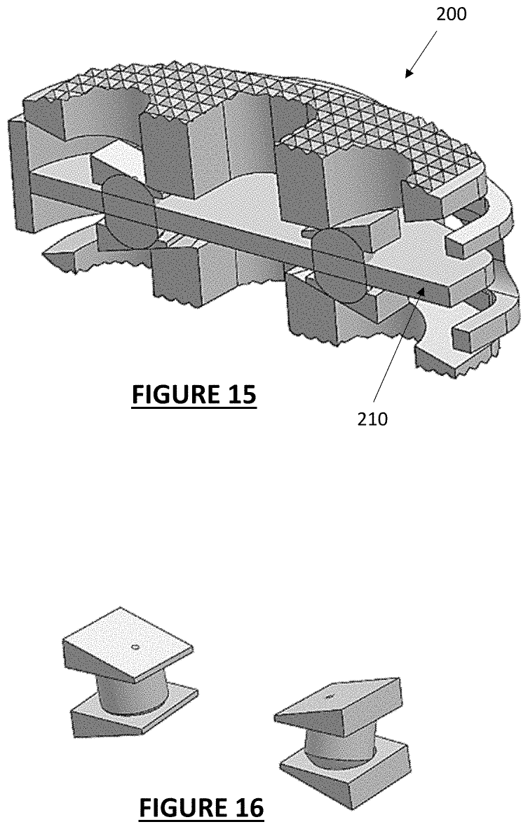

As mentioned, the example of FIGS. 1-10 is only an example and other examples are contemplated and are within the scope of the present application. FIGS. 11-18 are provided to emphasis this greater breath of scope (e.g., different mechanism/means for independently adjusting the distance between at least some of the respective engagement areas). FIGS. 11-14 are provided to generically show mechanism/means for cause the wedge on tapered surface can be something other than thread-on-thread engagement. FIG. 15 is provided to generically shown that actuation and/or adjustability could be achieved by mechanisms other than an elongated member. Specifically, a plate-like member 210 could be used as part of an actuator to adjust the height and/or angulation of the various implant surfaces, such as by moving the plate horizontally relative to other implant components. FIG. 15 is a partially torn-open perspective view of an implant device 200 in accordance with at least one aspect of the present subject matter, and shows another somewhat generic rendering of means/mechanism (e.g., including a plate member) that is operable to independently adjusting a distance between at least some of respective engagement areas of the two engagement members one relative position of two engagement members, with the generic rendering to shown that other/various means/mechanism are contemplated and are within the scope of the present application. Is such alternative examples, the plate can be moved to cause a translation of the motive force to adjust the distance between at least some of the respective engagement areas.

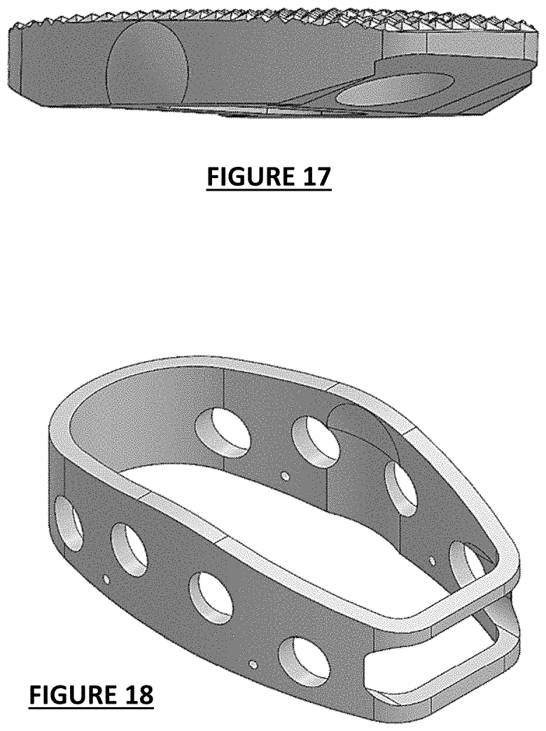

FIG. 16 is provided to generically show that the structure of the wedges and balls can be varied to some other structure/configuration. In general, one type of motive force/direction can be changed/translated to another type of motive force/direction. For example, FIG. 16 is somewhat schematized perspective view to show that different wedge members and/or balls that can be used within the means/mechanism is operable to independently adjusting a distance between at least some of respective engagement areas of the two engagement members. For example, different wedge angles, elongated (e.g., ovoid-shaped) balls are possible. FIG. 17 is provided to generically shown that the structure of the engagement member(s) can be varied.

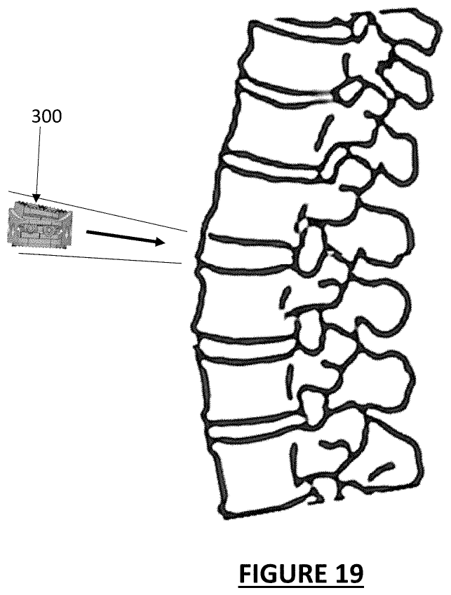

The various embodiments of an implant device 300 can be configured to interact with two bone vertebrae of a spine. An example of this interaction is shown in FIG. 19. As mentioned above, the spine may have any of several types of spinal curvature disorders. Examples of such spinal curvature disorders include, but need not be limited to, lordosis, kyphosis and scoliosis.

In one example scenario, the implant device can fix and secure adjacent vertebrae that have had cartilaginous disc between the vertebrae replaced with fusion material that promotes the fusion of the vertebrae, such as a graft of bone tissue. Also, such can be accomplished even when dealing with a spinal curvature disorder (e.g., lordosis, kyphosis and scoliosis).

Of course, method(s) for manufacturing the implant device and implanting the device into a spine (see FIG. 19) are contemplated and are part of the scope of the present application.

Again, variations, etc. are contemplated and are part of the scope of the present application. As examples, please note the following:

An implant device for the spine. The implant device is for location between two adjacent vertebrae. The implant device includes: a first engagement member configured to interface with a first of the two adjacent vertebrae, the first engagement member including plural engagement areas; a second engagement member configured to interface with a second of the two adjacent vertebrae, the second engagement member including plural engagement areas, distance exists between respective engagement areas of the first and second engagement member; and means for independently adjusting the distance between at least some of the respective engagement areas.

An implant device for the spine. The implant device is for location between two adjacent vertebrae. The implant device includes: a first engagement member configured to interface with a first of the two adjacent vertebrae, the first engagement member including plural corner areas; a second engagement member configured to interface with a second of the two adjacent vertebrae, the second engagement member including plural corner areas, distance exists between respective corner areas of the first and second engagement member; and means for independently adjusting the distance between at least some of the respective corner areas.

An implant device for the spine. The implant device is for location between two adjacent vertebrae. The implant device includes: a first engagement member configured to interface with a first of the two adjacent vertebrae, the first engagement member including plural side areas; a second engagement member configured to interface with a second of the two adjacent vertebrae, the second engagement member including plural side areas, distance exists between respective side areas of the first and second engagement member; and means for independently adjusting the distance between at least some of the respective side areas.

An implant device for the spine, the implant device for location between two adjacent vertebrae, the implant device being adaptive to address at least one spine curvature disorder. With one example, the at least one spine curvature disorder includes Lordosis.

An implant device for the spine, the implant device for location between two adjacent vertebrae, the two adjacent vertebrae have opposed faces that have at least one opposing inconsistency, the implant device being adaptive to address the at least one opposing inconsistency.

An implant device for the spine. The implant device is for location between two adjacent vertebrae. The implant device includes: a first engagement member configured to interface with a first of the two adjacent vertebrae, the first engagement member including plural engagement areas; a second engagement member configured to interface with a second of the two adjacent vertebrae, the second engagement member including plural engagement areas, distance exists between respective engagement areas of the first and second engagement member; and at least one mechanism that is operable to independently adjusting the distance between at least some of the respective engagement areas. Within one example, the at least one mechanism includes at least one screw.

A method for manufacturing an implant device as indicated above.

A method for manufacturing an implant device as set for within any of the details described with the present application.

An implant device for the spine as set for within any of the details described with the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

1. An implant device for the spine, the implant device for location between two adjacent vertebrae, comprising:
a first engagement member configured to interface with a first of the two adjacent vertebrae, the first engagement member including a plural of engagement areas;
a second engagement member configured to interface with a second of the two adjacent vertebrae, the second engagement member including a plurality of engagement areas,
an adjustment mechanism positioned at least partially between the first and second engagement members, the adjustment member capable of adjusting an axial separation distance between the first and second engagement members to alter a height of the implant device, the adjustment member further capable of adjusting a medial-lateral tilt-angle and an anterior-posterior tilt angle of the first engagement member relative to the second engagement member.

2. The implant device of claim 1, wherein the adjustment member is configured to adjust a medial-lateral tilt-angle and an anterior-posterior tilt angle of the first engagement member relative to an actuator of the adjustment mechanism.

3. The implant device of claim 1, wherein the adjustment member comprises a plurality of rotatable adjustment screws.

4. The implant device of claim 3, wherein the adjustment member comprises a pair of spaced apart rotatable adjustment screws.

5. The implant device of claim 3, wherein the adjustment member comprises a first rotatable adjustment screw having a first longitudinal axis and a second rotatable adjustment screw having a second longitudinal axis, and rotation of at least one of the first and second rotatable adjustment screws alters an alignment between the first and second longitudinal axes.

6. The implant device of claim 1, further comprising a centrally positioned skirt which fully surrounds a periphery of at least one of the first and second engagement members.

7. The implant device of claim 6, wherein the centrally positioned skirt includes at least one lateral opening which provides access to the adjustment mechanism.

8. The implant device of claim 1, wherein:
the first engagement member includes a first generally planar engagement surface having a first plurality of corner areas;
the second engagement member includes a second generally planar engagement surface having a second plurality of corner areas; and
adjustment means for independently adjusting the distance between at least some of the first and second pluralities of corner areas.

9. An implant device for the spine, the implant device for location between two adjacent vertebrae, the implant device comprising:
a first engagement member configured to interface with a first of the two adjacent vertebrae, the first engagement member including plural side areas;
a second engagement member configured to interface with a second of the two adjacent vertebrae, the second engagement member including plural side areas, distance exists between respective side areas of the first and second engagement members; and
means for independently adjusting the distance between at least some of the respective side areas.

10. The implant device of claim 9, wherein the implant device us implanted between two adjacent vertebrae, the implant device being adaptive to address at least one spine curvature disorder.

11. The implant device as set forth in claim 10, wherein the at least one spine curvature disorder includes Lordosis.

12. An implant device for the spine, the implant device for location between two adjacent vertebrae, the implant device comprising:
a first engagement member configured to interface with a first of the two adjacent vertebrae, the first engagement member including plural engagement areas;
a second engagement member configured to interface with a second of the two adjacent vertebrae, the second engagement member including plural engagement areas, distance exists between respective engagement areas of the first and second engagement members; and
at least one mechanism that is operable to independently adjusting the distance between at least some of the respective engagement areas.

13. The implant device as set forth in claim 12, wherein the at least one mechanism includes at least one adjustment screw.
